(19) United States
(12) Patent Application Publication
(10) Pub. No.: US 2010/0292764 A1
(43) Pub. Date: Nov. 18, 2010

(54) LAPAROSCOPIC KIDNEY COOLING SHEATH

(75) Inventors: Naeem Soomro, Newcastle upon Tyne (GB); Clive Griffiths, Newcastle upon Tyne (GB); Elaine Colechin, Newcastle upon Tyne (GB); John Riddle, Newcastle upon Tyne (GB)

Correspondence Address: Zilka-Kotab, PC, P.O. BOX 721120, SAN JOSE, CA, 95172-1120, US

(73) Assignee: The Newcastle Upon Tyne Hospitals NHS Trust, Newcastle upo Tyne (GB)

(21) Appl. No.: 12/298,885

(22) PCT Filed: Apr. 26, 2007
(86) PCT No.: PCT/GB07/01526

§ 371 Date: Aug. 9, 2010

(30) Foreign Application Priority Data
Apr. 28, 2006 (GB) 0608419.8

(51) Int. Cl. A61F 7/12

(52) U.S. Cl. 607/105

(57) ABSTRACT

A device (100) and method for achieving localised hypothermia during laparoscopic surgery comprises a sheath portion (101) in the form of a flexible bag-like shape which is shaped and sized so that it substantially surrounds the organ of interest in use. The sheath portion (101) comprises walls, each consisting of at least a first inner layer and a second outer layer which define a space therebetween into which catheters (104a, b) deliver and remove cooled fluid to provide a cooling effect to the organ.

The present invention relates to the field of laparoscopic surgery, more specifically partial nephrectomy (LPN), and in particular to an improved means for achieving localised hypothermia which results in substantially uniform renal hypothermia, and which is quick and simple to use, and application of said means.

In recent years laparoscopic partial nephrectomy (LPN) has emerged as a viable alternative option to open surgery in the treatment of small (less than 4 cm) renal cell carcinoma. While laparoscopic partial nephrectomy benefits from the advantages inherent to all laparoscopic techniques; such as reduced morbidity and shorter recovery times, the technical complexity associated with the need to secure hemostasis and to achieve renal hypothermia, has largely limited LPN to the treatment of small or polar lesions.

During the LPN procedure, transient hilar control is typically exercised. This temporary arterial occlusion decreases blood loss during surgery as well as providing a substantially bloodless field in which the accurate excision of the tumour can be performed. However, obstruction of the inflow of arterial blood can lead to a corresponding ischaemia in the organ which can result in permanent kidney damage after a certain length of time. The maximum tolerable period of warm ischaemia has previously been reported as 30 minutes in humans (Novick A C. 1983; Urol Clin North Am, 4:637-644 “Renal hypothermia: in vivo and ex vivo”; McDougal W S. 1988; J Urol, 140:1325-1330 “Renal perfusion/reperfusion injuries”.)

The induction of renal hypothermia, in which renal parenchymal temperature is lowered, can protect the kidneys from ischaemic damage sustained during periods of arterial occlusion. Lowering the local temperature at the site of restricted blood flow reduces the metabolic activity of cortical cells, leading to reduced oxygen consumption and reduced ATP breakdown. This in turn leads to a concomitant increase in the maximum tolerable ischaemia period. Of great importance in the induction of local hypothermia is the attainment of a uniform hypothermia throughout the kidney surface, cortex and medulla. A uniform temperature of 20-25° C. (Ramani 2006; B J U Int, 97:342-344 “Current concepts in achieving renal hypothermia during laparoscopic partial nephrectomy”) has proven adequate to allow periods of up to 3 hours of temporary arterial occlusion to be tolerated by the kidney, with no permanent damage ensuing. This extended ischaemia time effectively extends the time available to the surgeon in which to perform the partial nephrectomy procedure. Although laparoscopic renal hypothermia has been achieved through a variety of techniques, these techniques have thus far failed to duplicate open surgery methods and have proved cumbersome and complicated to perform.

Methods of cooling the kidney during open surgery involve techniques which exploit the large access area to the kidney, such as ice-filled bags or cooling jackets. Cooling jackets known in the art for the containment of body organs such as the heart or kidney during open surgery comprise generally flat pads having an interior compartment through which a coolant is circulated, through pre-defined fluid channels. In use, these jackets are inserted into the body via the large surgical opening, and placed under the organ, before being wrapped around it to the front. The opposite sides of the jacket are then overlapped and secured with tabs. However, these jackets were designed with open surgical methods in mind and are thus too large and cumbersome to be suitable for use with minimally invasive keyhole surgery techniques (laparoscopic surgery).

Advances in laparoscopic techniques have necessitated new techniques for the induction of renal hypothermia which are compatible with the keyhole surgical approach. Various techniques have been described in the art for inducing laparoscopic renal hypothermia. The technique of laparoscopic ice-slush renal hypothermia was first described by Gill et al. in 2003 (Gill 2003; J Urol, 170:52-56 “Laparoscopic ice slush renal hypothermia for partial nephrectomy; the initial experience”). This study described the insertion of a 15-mm Endo Catch II™ bag (single-use specimen pouch) through a laparoscopic incision. The bag was opened and positioned around a kidney, and clips were used to fix the mouth of the bag around the renal hilum. The bottom end of the bag was then withdrawn outside of the body, secured and cut open. Sterile ice-slush, which had been manually stirred to a fine consistency, was then inserted into the bag via pre-loaded syringes to completely surround the kidney with ice. 30 cc syringes had been modified by cutting off the nozzle end to facilitate rapid injection of the ice slush and thus extension of the port site incision by 2 to 3 mm was required to facilitate the insertion of the syringe barrel. The open end of the bag was then closed with a tie, the bag reinserted into the abdomen, and pneumoperitoneum restored. A laparoscopic sponge was positioned around the bag to prevent the bowel coming into contact with the ice-filled bag. After approximately 10 minutes of cooling, the bag was incised and ice slush was removed from around the tumour site, leaving the ice in contact with the remainder of the kidney surface. LPN was then performed using standard techniques.

The use of laparoscopic ice-slush to achieve renal hypothermia was also described by Wakabayashi et al. (Wakabayashi 2004; Urology, 63(4):773-775 “Renal hypothermia using ice slush for retroperitoneal laparoscopic partial nephrectomy”). This technique necessitated the extension of the primary port incision to facilitate the insertion of a cylindrical insertion device which was approximately 3 cm in diameter at the insertion end. Approximately 700 ml of ice slush was introduced into the retroperitoneal space and distributed evenly around the kidney. The authors indicated that although the ice slush was placed in direct contact with the retroperitoneal space, direct complications of adjacent organs did not occur. However, it is commonly understood that direct contact of surrounding organs with ice or ice slush is not desirable.

It has therefore been demonstrated that these ice slush techniques result in adequate cooling of the kidney. However, these techniques expose the entire surgical field and surrounding organs and structures to the effect of the ice. Furthermore, the confined working space can make surgical procedures, such as tumour excision difficult when the retroperitoneal area is filled with ice slush, and visibility of the area is also typically impaired by the presence of ice. Furthermore, the ice slush must be partially or completely removed to operate, which can be both time-consuming and messy to perform. Recooling, which is often necessary during procedures after 20-30 minutes is also complicated to perform, often requiring the insertion of fresh ice slush, and its removal, before surgery can be resumed. A still further drawback of this technique is the risk of ice-slush escaping or leaking into the abdominal cavity.

A cooling sheath for use in laparoscopic techniques has been described by Herrell et al. (Herrell et al 1998; J. endourology, 12(2): 155-161 “The laparoscopic cooling sheath: a novel device for hypothermic preservation of the kidney during temporary renal artery occlusion”). This sheath comprised a cooling jacket with integrated fluid paths which was inserted via laparoscopic methods, and manoeuvred into position around the kidney. In the first insertion method, the sheath was introduced through a 4 cm open incision site with a pursestring suture, whilst in an alternative method the sheath was rolled up and inserted into the body cavity via an 18 mm trocar. A heat exchanger pumped fluid around the jacket to provide cooling to the organ. Whilst this jacket overcame some of the disadvantages associated with previous ice-slush techniques, it failed to overcome difficulties associated with laparoscopic insertion and the jacket was later described, in a paper co-authored by its own inventor, as “complex to manufacture and apply” (Webster T. M. et al. 2005; J. Endourology, 19(9): 1075-1081 “Simple method for achieving renal parenchymal hypothermia for pure laparoscopic partial nephrectomy”).

The present invention identifies the drawbacks of known kidney cooling devices and methods and proposes an improved means for achieving laparoscopic renal hypothermia which mitigates one or more of the limitations previously addressed.

The aims and objects of the invention will become apparent from reading the following description.

According to a first aspect of the present invention there is provided a laparoscopic cooling device for lowering the temperature of an organ comprising:
a sheath, the walls of the sheath comprising a first inner layer and a second outer layer which define a space therebetween;
at least one coolant transport tube adapted to transport coolant into the space defined by the first inner layer and the second outer layer;
   wherein at least one of the layers is deformable to the extent that it will conform to the surface of the organ when the space defined by the first inner layer and the second outer layer contains coolant.

Advantageously, the first inner layer of the sheath which comes into contact with an organ during use is sufficiently flexible and deformable such that it conforms to the surface contours of the organ of interest when coolant is present. This ensures that the sheath provides a uniform cooling effect across the surface of the organ as the entire surface, or a significant portion thereof, is in contact with the sheath. When there is no coolant present, less pressure is exerted against the deformable layer ensuring that it does not conform to any particular shape, such that it can easily be inserted through a very small aperture.

Previous cooling devices have typically been in the form of a rigid sleeve or jacket, which surround the organ of interest. As organs can vary greatly in size, and even similar organs can be quite different in size, the cooling effect of the cooling sleeve or jacket is often compromised at the edges of the organ or indeed for smaller organs, which may not come into sufficiently close contact with the cooling device to provide the requisite cooling effect. Furthermore, there are often surface contours on an organ, such that even relatively snug-fitting jackets as described previously do not uniformly cover the surface. However, for the laparoscopic cooling device of the invention, as the first inner layer of the sheath conforms to the surface contours of the organ of interest, the sheath is suitable for use with organs of different sizes and shapes.

Advantageously, the flexibility of the sheath of the invention allows the sheath to be compressed and inserted into the body using an introducer of small diameter. Typically, the sheath of the present invention can be introduced through an 18 mm trocar or introducer, although in many cases the sheath can be introduced through even smaller trocars or introducers, having diameters of between 5-15 mm.

A further advantage of the sheath is that it can be folded and unfolded easily and quickly due to its flexible nature to facilitate insertion during the laparoscopic procedure.

Optionally the laparoscopic cooling device further comprises a shaft portion.

The shaft portion is essentially a stem which is positioned at one end of the sheath and which acts to locate the coolant transport tube or tubes in one area. Ideally, the shaft is at least semi-rigid to ease the insertion of the sheath through a trocar traversing a surgical incision. However, it can be beneficial to avoid including a shaft portion as this lessens the number of components present in the body during laparoscopic surgery.

Optionally the walls of the sheath comprise a third layer.

Preferably, if provided, the third layer is located external to the outer layer and defines one or more cavities therebetween.

Preferably, the cavity between the third layer and the second outer layer is inflatable when air is provided thereto.

Advantageously, the inflatable cavity facilitates the opening up of the sheath when inside the body. Once the sheath is unfurled it can be manipulated into position surrounding the organ of interest. The inflatable cavity can then be deflated to allow the sheath to be secured around the organ.

A further advantage of the inflatable cavity is that it can be re-inflated subsequent to the securing of the sheath around the organ. The inflatable cavity then provides a thermal insulation barrier between the third layer and the coolant-containing space between the first inner layer and the second outer layer. This allows the cooling of the organ to be effected more efficiently as temperature loss to surrounding tissues is minimised. Consequently the time taken to achieve the desired level of cooling can be reduced. This also reduces the cooling of nearby organs.

Preferably, if provided, the third layer is connected to the second outer layer via a series of vertical welds.

The term vertical means substantially parallel to the shaft portion.

Preferably the vertical welds are discontinuous.

The effect of the vertical welds is to compartmentalise the inflatable cavities forming a series of inflatable pockets. As the vertical welds are discontinuous, they allow the air to move through the pockets ensuring uniform inflation of the cavity. The vertical welds further help to maintain the overall shape of the device.

The sheath may be substantially rectangular in shape.

Preferably, the sheath comprises an elongate tail portion.

Advantageously, the elongate tail portion can protrude from the trocar or introducer during use. The tail portion can thus conveniently be used to facilitate removal of the sheath from the patient's body when required.

Preferably, the sheath comprises a bag.

The term “bag” is taken to mean an at least partially flexible container or receptacle comprising an open end or mouth; or something resembling or suggesting such a container.

Preferably, the sheath comprises a first open end.

Preferably, the sheath comprises a bag having a first open end, whereby in use, the bag surrounds an organ of interest.

Providing a first open end of the sheath allows the sheath to be manoeuvred to surround the organ of interest at which point the first open end can be closed about the organ so that it is substantially contained within the sheath.

The orientation of the first open end of the sheath facilitates the manoeuvring of the sheath into position around the organ of interest.

The first open end of the sheath may lie in a plane perpendicular to the longitudinal axis of the device.

Preferably, the sheath is 15 cm in length across its first open end.

Optionally, the sheath is between 12 cm and 18 cm in length across its first open end.

Preferably, the sheath has a depth of 15 cm.

The sheath needs to be big enough to easily fit around the organ in question, including any surrounding fat and the tumour itself. Moreover, the sheath has to be big enough such that there is enough room available to introduce fluid into the space between the inner and outer layers when the sheath is in situ around the organ; and also to introduce air where a third layer is present in the sheath. In the case of a kidney, a sheath length of 15 cm with a depth of 15 cm has been found to be the most advantageous size. However, different sheath sizes are envisaged for alternative organs or patients.

Preferably the sheath is formed of a plastics material.

Alternatively, any material having suitable thermal properties and which can be rolled up into small dimensions can be used, for example tin-coated foils.

Preferably, the laparoscopic cooling device comprises a plurality of coolant transport tubes.

More preferably, the laparoscopic cooling device comprises a first coolant transport tube and a second coolant transport tube wherein the first coolant transport tube is configured to deliver coolant into the space between the first inner layer and the second outer layer and the second coolant transport tube is configured to remove coolant from the space. Optionally these coolant transport tubes may be supplemented by additional coolant delivery or coolant removal tubes.

Optionally, the coolant transport tubes are integrated into the second outer layer of the sheath.

Preferably, in use a supply of coolant is delivered into the space and the coolant currently in the space is removed such that a constant cooling effect is provided by the sheath.

Preferably, the coolant transport tube or tubes are formed of a flexible material.

The flexibility of the coolant transport tubes further increases the pliability of the laparoscopic cooling device such that it can be more easily folded or rolled up to facilitate insertion through an introducer.

The flexible tube or tubes may be tubular catheters or may be other suitable forms of flexible tubing.

Optionally the flexible tube or tubes may be integrated into the outer sheath.

Preferably the coolant transport tubes are connected to an external cooling system.

Preferably the external cooling system is adapted so that the cooling fluid flows in a closed loop.

Advantageously, ensuring that the cooling fluid flows in a closed loop reduces the chances of infection.

Preferably, the external cooling system comprises a heat exchanger and a pump.

More preferably, the external cooling system further comprises a flow rate adjustment module.

Providing a flow rate adjustment module within the external cooling system allows the flow of coolant into the space to be adjusted. This can be employed to speed up or slow down the rate of introduction of fresh coolant into the space such that a desired constant temperature can be maintained.

Still more preferably, the external cooling system further comprises a temperature monitoring element.

It has previously been disclosed that the maintenance of the organ temperature between an established range can reduce deleterious ischemic effects associated with the restriction of the flow of blood to the organ. Therefore, providing a temperature monitoring element within the external cooling device allows medical personnel to monitor the temperature throughout the surgical procedure to ensure that the organ of interest is maintained at the requisite temperature for the duration of the laparoscopic surgery.

Preferably, the temperature monitoring device is also configured to monitor the temperature of the coolant within the space.

Optionally, the temperature monitoring device is configured to monitor the temperature of one or more surfaces of the sheath or the surface of the organ of interest.

Preferably, a narrow channel is provided at the border of the open end of the sheath.

Preferably, the narrow channel is defined by a seam connecting the first inner layer and the second outer layer of the sheath to define a pathway which encircles the open end of the sheath.

Preferably, the laparoscopic cooling device comprises means for manoeuvring the sheath to surround the organ of interest.

Optionally the sheath comprises at least one rigid or semi-rigid spine.

Preferably, the sheath comprises two rigid or semi-rigid spines.

Providing the sheath with a rigid or semi-rigid spine allows the sheath to be more readily guided into position within the body as the rigid or semi-rigid spine can be manoeuvred beneath the organ of interest to more easily position the sheath therearound.

Preferably the rigid or semi-rigid spine comprises a plastic rod.

Preferably, the sheath further comprises at least one rigid or semi-rigid tube.

Preferably, the sheath comprises two rigid or semi-rigid tubes.

Preferably, the at least one rigid or semi-rigid tube is provided within the space defined by the first inner layer and the second outer layer.

Optionally, the at least one rigid or semi-rigid tube is provided within the cavity defined by the third layer and the outer layer where provided.

Preferably, the at least one rigid or semi-rigid tube is configured to form the rigid spine of the sheath.

Preferably the at least one rigid or semi-rigid tube is a narrow plastic tube or tubes.

Alternatively the at least one rigid or semi-rigid tube is a narrow brass tube.

Preferably the at least one rigid or semi-rigid tube extends from the shaft portion of the laparoscopic cooling device to the narrow channel at the edge of the sheath.

Preferably, the at least one rigid or semi-rigid tube enters the narrow channel at the border of the sheath via an access point in the seam joining the first inner layer and the second outer layer.

Most preferably, a coupling or couplings form a seal between the rigid tube and its point of entry to the narrow channel at the border of the sheath to create an airtight channel.

Preferably the laparoscopic cooling device further comprises means for closing the open end of the sheath to surround the organ of interest.

Preferably the means for closing the open end of the sheath to surround the organ of interest is operable or partially operable outside of a patient's body.

Preferably, the laparoscopic cooling device comprises a drawstring.

Preferably, the drawstring is provided within the narrow channel at the border of the sheath.

Most preferably, the drawstring is provided encircling the narrow channel at the border of the sheath, and along the tail portion of the sheath so that the end(s) of the drawstring are provided outside of the patient's body in use.

Advantageously, as the ends of the drawstring are provided outside of the patient's body when the laparoscopic cooling device is in use, this means that the drawstring can be operated outside of the patient's body. This avoids the need to secure tabs or fasteners within the patient's body in order to position or fix the sheath around the organ of interest. This is particularly advantageous as the confines of the retroperitoneal areas, accessible via keyhole incisions, can make the operation of fastening devices within the patient's body both difficult and time-consuming to perform.

Optionally, the drawstring is provided within at least one of the semi-rigid tube or tubes.

Optionally, the drawstring is provided encircling the narrow channel at the border of the sheath, through the rigid or semi-rigid tube and along the length of the shaft portion of the laparoscopic cooling device, so that the end(s) of the drawstring are provided outside of the patient's body in use.

Preferably, the drawstring comprises a locking means.

Advantageously, the locking means is variably positionable along the drawstring such that it prevents the drawstring from loosening.

Preferably, the locking means is deformable.

Most preferably, the locking means comprises a deformable rubber member.

Optionally, the locking means comprises a toggle.

Alternatively, the locking means comprises a releasable cable tie.

As a further alternative the locking means comprises a ratchet and pawl-type mechanism.

Preferably the laparoscopic cooling device comprises means to facilitate the opening out of the open end of the sheath inside the patient's body.

Facilitating the opening out of the open end of the sheath inside the patient's body allows the sheath to more readily be manoeuvred into position surrounding the organ of interest. Furthermore this allows the sheath to be tightly rolled or bundled up prior to insertion into the patient's body.

Optionally, the means to facilitate the opening out of the open end of the sheath comprises the provision of CO₂ to the airtight channel.

Alternatively, the means to facilitate the opening out of the open end of the sheath comprises the provision of air to the airtight channel.

Preferably the laparoscopic cooling device comprises means for facilitating the insertion of the device through an introducer or trocar.

Preferably, the means for facilitating the insertion of the device through an introducer or trocar is a rigid overtube.

Optionally, the rigid overtube may be made in situ by rolling a sheet of stiff material around the sheath.

Preferably the stiff material is transparent.

More preferably the stiff material is an acetate sheet.

The rigid overtube can then be slipped over the rolled sheath and the shaft portion of the laparoscopic cooling device if present, to allow the sheath to be maintained in a folded position during insertion. As the sheath does not conform to any particular shape when there is no coolant or air contained therein, the sheath can easily be folded tightly and compressed into the overtube. The overtube makes a seal with the introducer or trocar and facilitates insertion therethrough. The overtube keeps the sheath in a tightly bundled conformation in order to allow insertion through the smallest possible introducer. Once the rigid overtube has been inserted into the trocar or introducer, it is removed from the trocar or introducer, leaving the remainder of the device behind.

Advantageously, the drawstring can be pulled in order to close the sheath either before or after the removal of the rigid overtube from the trocar or introducer.

Optionally, the laparoscopic cooling device may comprise means to partition the sheath when in position surrounding the organ of interest.

Preferably the means to partition the sheath is reversible.

Providing means to partition the sheath advantageously allows surgical access to a chosen site, for example a tumour site, to enable the necessary surgical procedure to be carried out whilst contact is maintained between the remainder of the organ surface and the partitioned sheath.

Preferably the means to partition the sheath when in position surrounding the organ of interest comprises a rip cord.

Preferably the rip cord is provided in the form of a tearable seam connecting the first inner layer and the second outer layer which is deployable to partition the sheath into two or more sections.

Optionally the means to partition the cooling sheath comprises a zip.

Preferably, the organ of interest is the kidney.

Preferably, the laparoscopic cooling device is for use during and/or before laparoscopic partial nephrectomy.

Optionally, the laparoscopic cooling device may include a thermocouple or thermocouples.

The laparoscopic cooling device may comprise a thermocouple or thermocouples for the purpose of measuring the temperature of the organ of interest.

At least one thermocouple junction may be provided at a surface of the sheath.

Optionally, at least one thermocouple junction is provided at the surface of the first inner layer which comes into contact with the organ of interest.

Optionally, at least one thermocouple junction is integrated into the sheath.

The advantage of providing at least one thermocouple junction at the surface of the sheath is that the temperature of the organ surface can be monitored during the laparoscopic procedure. For example, when the organ of interest is the kidney, thermocouple junctions can be located in contact with the upper and lower poles of the kidney in order to measure the temperature of the kidney before, during or after the surgical procedure.

Optionally, a thermocouple or thermocouples are provided for the purpose of measuring the temperature of the fluid upon delivery into or removal from the sheath.

Optionally, the thermocouple can use the same point of introduction as the laparoscopic cooling device.

Optionally, a thermocouple or thermocouples can be provided externally as part of the heat exchanger system.

Optionally, at least one thermocouple junction is provided on the coolant transport tubes.

Providing one or more thermocouples junctions on the coolant transport tubes allows the temperature of the coolant in the space between the first inner layer and the second outer layer of the sheath to be monitored.

Preferably, the laparoscopic cooling device comprises markings to aid with depth perception.

Providing markings along the edges of the laparoscopic cooling device assist in the surgeon's depth perception when viewing images of the abdominal cavity during the procedure.

Preferably the marking are provided along the circumference of the open end of the sheath.

Preferably, the markings are provided in one or more different colours.

Preferably the markings are provided in two different colours.

According to a second aspect of the invention there is provided a method of cooling an organ for surgery using the laparoscopic cooling device of the first aspect comprising:
inserting the sheath into a body cavity
employing means to facilitate the opening out of the open end of the sheath inside the patient's body
manipulating the sheath to surround the organ of interest
securing the sheath around the organ of interest
providing coolant to the sheath

The means to facilitate the opening out of the open end of the sheath inside the patient's body may be the provision of gas or fluid to the sheath to cause it to inflate.

Preferably, the sheath is arranged into a tight bundle before insertion into the body cavity.

Optionally, after the sheath is arranged into a tight bundle, it is introduced into the body through an introducer.

Optionally, after the sheath is arranged into a tight bundle, a stiff sheet is wrapped around the bundle.

The stiff sheet forms a rigid overtube around the bundled sheath, keeping the sheath neatly rolled. This facilitates the insertion of the sheath through an appropriate trocar or introducer into the body cavity. Once the rigid overtube has been inserted into the trocar or introducer it is removed therefrom, leaving the remainder of the device behind.

Optionally air is provided to the sheath to cause it to inflate.

An alternative option is that CO₂ is provided to the sheath to cause it to inflate.

Providing air or CO₂ to the sheath causes it to unfurl within the body cavity. This facilitates the manipulation of the sheath to surround the organ.

Preferably the sheath is secured around the organ of interest using a drawstring.

Preferably the method comprises the step of monitoring the temperature of the organ of interest during the surgery.

Optionally the method comprises the step of monitoring the temperature of the coolant during the surgery.

A preferred embodiment of the invention will now be described with reference to the accompany drawings in which:

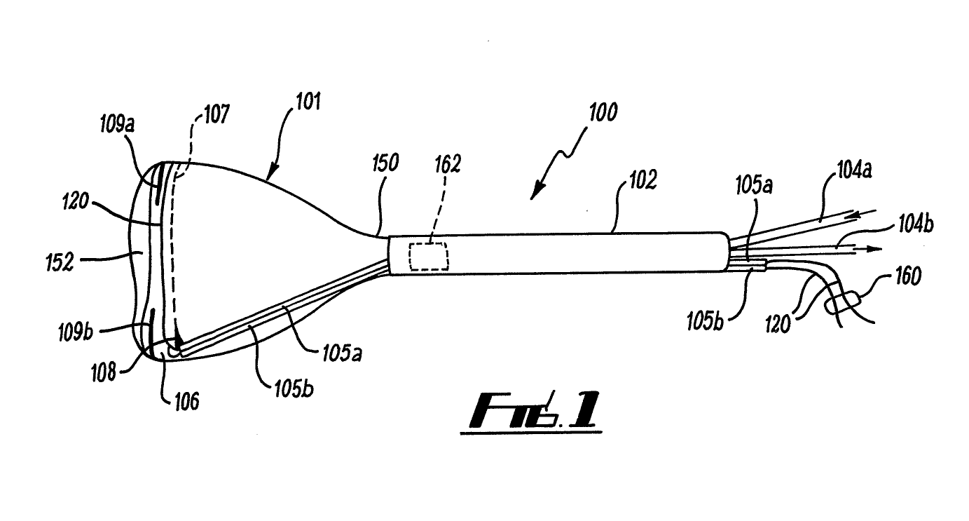

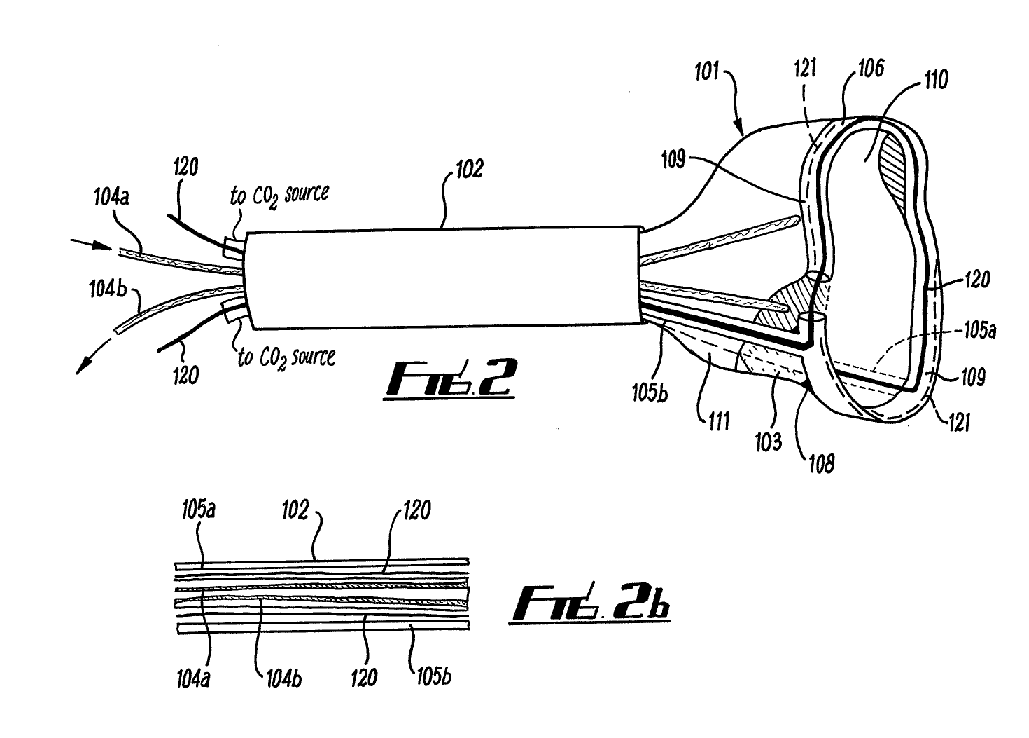

FIG. 1 illustrates a laparoscopic cooling device according to a first embodiment of the invention.

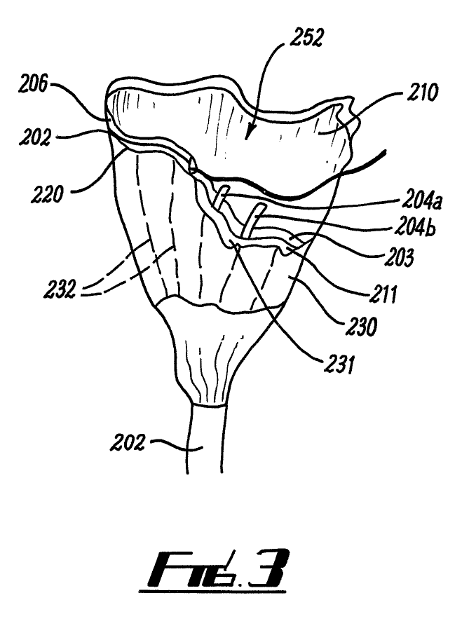

FIG. 2 illustrates a sheath in more detail according to a first embodiment of the invention.

FIG. 2b is a cross-section view of the shaft portion of FIG. 2.

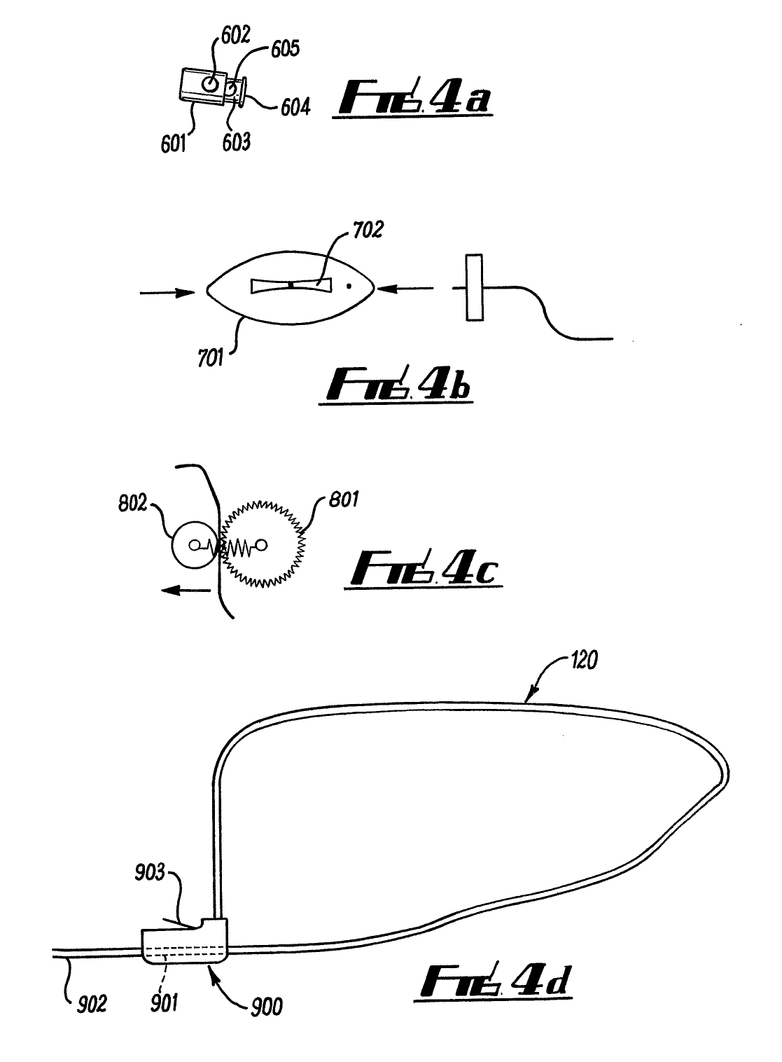

FIG. 3 illustrates a laparoscopic cooling device according to an alternative embodiment of the invention.

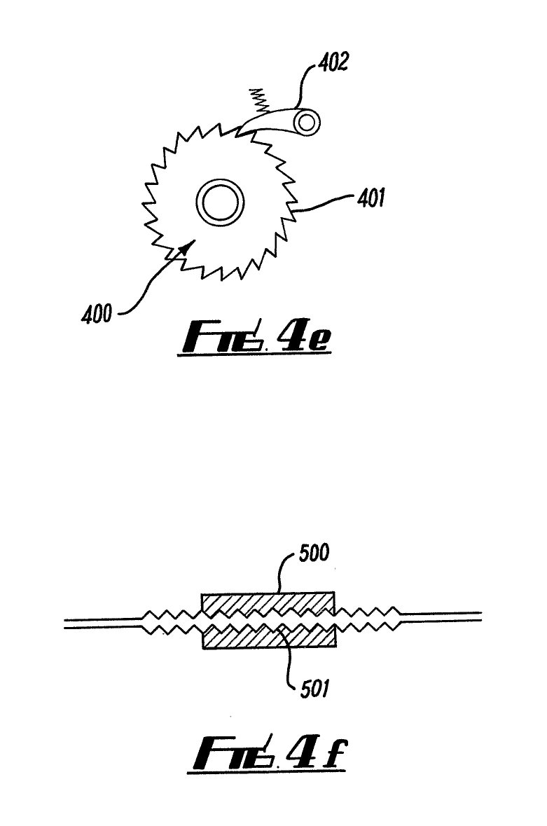

FIGS. 4(a)-4(f) illustrate a series of locking means, suitable for use with the laparoscopic cooling device of the invention.

Turning firstly to FIG. 1, the drawing shows a laparoscopic cooling device for use in laparoscopic partial nephrectomy according to an embodiment of the invention. The device is generally depicted at 100. The laparoscopic cooling device 100 comprises a sheath portion which is indicated generally at 101, and a shaft portion which is fixed to the sheath and which is indicated at 102. The sheath 101 is of a generally rectangular shape, with a tail portion 150, a part of which extends through the shaft portion 102. The tail portion 150 is sized and shaped so that it remains outside of the trocar (and therefore also the body) in use. The sheath 101 is a bag-like structure which is shaped and sized so that it substantially surrounds the organ of interest, in use. In this way, the whole surface area of the organ of interest is able to be cooled when required. The depicted sheath 101 comprises an opening 152, i.e. a portion of the sheath that is open to allow the sheath to be placed around the organ of interest. In the depicted embodiment, the opening 152 of the sheath 101 is a side opening, whereby it is oriented in a plane perpendicular to the longitudinal axis of the laparoscopic cooling device 100. The sheath 101 also comprises a small tail opening (not shown) at the tail portion 150 of the sheath 101, which allows access to the sheath. The sheath portion 101 comprises walls, each consisting of a first inner layer and a second outer layer which define a space therebetween. Tubular catheters 104a,b are provided from an external cooling system (not shown) along the shaft portion 102 and into the space between the first inner layer and the second outer layer to deliver fluid coolant to and remove fluid coolant from the space in use. The tubular catheters 104a,b have a rounded tip to prevent accidental tearing of the sheath. In addition, each of the tubular catheters has at least four holes disposed at ninety degree intervals around the periphery of the catheter adjacent the distal end to permit fluid flow. The tubular catheters 104a,b are joined via Luer connectors (not shown) or similar, to the external cooling system which pumps fresh fluid coolant into the defined space via the inlet catheter 104a and removes warmed fluid coolant therefrom via the outlet catheter 104b.

The sheath 101 also houses two semi-rigid plastic tubes 105a,b which are positioned in the space between the first and second layers of the sheath and extend from outside the shaft portion 102, along the length thereof and then through the space between the first and second layers to a narrow channel 106 near the edge of the open end of the sheath portion 101. The semi-rigid plastic tubes 105a,b are substantially parallel to each other and are connected externally to a source of air via Luer connectors (not shown) or similar. The narrow channel 106 traverses the circumference of the open end 152 of the sheath 101 and is defined by a seam 107 joining the first and second layers of the sheath 101 and separating the narrow channel 106 from the main body of space between the first and second layers. In the depicted embodiment the narrow channel 106 of the sheath 101 is kept airtight by the use of couplings 108 which seal the narrow channel 106 against the semi-rigid plastic tubes 105a,b. Semi-rigid spines 109a,b in the form of plastic rods are provided within the narrow channel 106 to facilitate the positioning of the opening of the sheath 101 around the kidney. These semi-rigid spines 109a,b allow the sheath 101 to be scooped around the kidney, facilitating positioning of the sheath 101 in use. A drawstring 120 is provided along the length of the narrow channel 106, encircling the opening 152. The drawstring 120 runs along the semi-rigid plastic tubes 105a,b which traverse the space between the first and second layers and along the shaft portion 102, such that the ends of the drawstring 120 terminate outside of the shaft portion 102, and thereby outside of the patient's body in use. This allows the drawstring 120 to be operated outside of the patient's body in use. In particular, in use the drawstring 120 runs from outside of the patient's body, along the semi-rigid plastic tube 105a and into the narrow channel 106 circumventing the opening 152 of the sheath, and then back along the semi-rigid plastic tube 105b, to allow the sheath 101 to be closed around the organ of interest.

A locking means 160, which in this embodiment is in the form of a deformable rubber locking member (depicted at FIG. 4b), is provided at the free end of the drawstring 120, to allow the drawstring to be releasably secured once the sheath has been closed around the organ. A plastic clip 162 keeps the ends of the drawstring together in the tail portion 150 of the sheath 101.

Due to the flexible nature of the device 100, the sheath 101 can be rolled up and inserted into a rigid overtube. This enables the device to be rolled up into sufficiently small dimensions such that it can be inserted through an introducer or trocar. As soon as the sheath is pushed into the body of the patient and is freed from the confines of the rigid overtube and the trocar, the semi-rigid spines 109a,b open up the opening 152 of the sheath to allow the user to easily locate the sheath around the patient's kidney. Once the sheath is in position surrounding the organ, the sheath can be pulled closed by the drawstring 120.

Referring now to FIGS. 2 and 2b, the sheath 101 comprises walls consisting of a first layer 110 and a second layer 111 wherein the first layer 110 surrounds the organ of interest, in this example the kidney, in use. The first 110 and second layers 111 of the sheath 101 are substantially sealed to define a space 103 therebetween. Inlet and outlet tubular catheters 104a,b are provided which extend from the external cooling system (not shown) through the shaft portion 102 of the device and enter the space 103 via appropriate openings in the second layer 111 of the sheath 101. The tubular catheters 104a,b extend substantially through the length of the space 103 and are spaced apart to ensure even circulation of the coolant through the space 103 in use. The tubular catheters 104a,b, are joined via Luer connectors (not shown) or similar, to the external cooling system (not shown) which pumps fresh fluid coolant into the space 103 via the inlet catheter 104aand removes warmed fluid coolant therefrom via the outlet catheter 104b in a closed system.

A narrow channel 106 is further defined in the sheath 101 which circumscribes the opening 152 in the sheath 101. The narrow channel 106 is accessed via two semi-rigid plastic tubes 105a,b. These semi-rigid plastic tubes 105a,b are connected outside of the body via Luer connectors (not shown) or similar, to a source of CO₂. They then extend along the length of the shaft portion 102 and through the space 103 between the first 110 and second 111 layers to the narrow channel 106 at the border of the sheath 101.

Couplings 108a,b join the ends of the semi-rigid plastic tubes 105a,b with the entrance to the narrow channel 106, and prevent air from entering the channel, keeping it airtight. A drawstring 120 is further provided which extends from outside the body of the patient along the length of the shaft portion 102, through one of the semi-rigid plastic tubes 105a and along the narrow channel 106 and then back along the semi-rigid plastic tube 105b so that it ends outside of the patient's body. The drawstring 120 can be operated from outside of the patient's body to close the sheath 101 around the organ. Markings 121 along the edges of the sheath 101 assist in the surgeon's depth perception when viewing images of the abdominal cavity during the procedure. The markings 121 are provided along the circumferential edge of the sheath 101 following part of the path of the drawstring 120. The markings 121 are provided in two different colours, with a different colour being provided on each half of the circumference to assist in depth perception during insertion of the device. Clearly the markings 121 described in relation to this embodiment may also be useful in other embodiments of the invention.

In the depicted embodiment, the organ of interest is the kidney, although it will be understood that the inventive device is also suitable for use with alternative organs, for example the heart, liver or spleen. Adjustments to the dimensions of the device may be made in order to accommodate differently sized and shaped organs.

In use, a trocar is inserted into the body via an appropriate wound. The sheath 101 which is flexible and free of any coolant or air is rolled up to form a tight bundle. The bundled sheath 101 is then enclosed in an A4-sized acetate sheet (not shown), which is rolled around the bundled sheath 101 to form a rigid overtube. The acetate sheet and its contents are then inserted into the trocar and introduced into the body via the wound.

It is however to be understood that the rigid overtube may be a portion of tubing.

Once the rigid overtube has been inserted into the trocar or introducer, the sheath 101 is pushed further into the body relative to the acetate sheet and the trocar. The rigid overtube is then removed from the trocar or introducer, leaving the remainder of the device behind.

A pneumatic burst of air is then provided to the narrow channel, 106 via the semi-rigid plastic tubes 105a,b causing the narrow channel 106 to inflate gently, and thereby unfurling the sheath 101 within the abdominal cavity. The opening 152 in the sheath 101 can then be manoeuvred into position around the kidney using standard laparoscopic instruments inserted through other access ports. The semi-rigid spines 109a,b can assist in the positioning of the sheath 101, allowing the surgeon to “scoop” the organ into the open end of the sheath. Once the sheath 101 is in position, the drawstring 120 can be operated outside of the patient's body to help to close the open end 152 of the sheath 101 around the kidney. The drawstring 120 is then secured in position by deploying the locking means 160.

Fluid coolant is then provided to the space 103 via the inlet catheter 104a, which is connected to the external cooling unit (not shown). The fluid coolant fills the space 103, such that the flexible first layer 110 of the sheath 101, begins to deform so that it contours to the shape of the kidney surface. The outlet catheter 104b, pumps warmed fluid coolant from the space 103 so that cooling of the organ can be effected. A temperature monitoring element (not shown) and flow rate adjustment module (not shown) allow the temperature to be observed and controlled throughout the process.

An alternative embodiment of the invention can be envisaged, which is illustrated in FIG. 3. In this embodiment, the sheath 201 comprises three layers 210, 211 and 230. A space 203 is defined between the first inner layer 210, which surrounds the kidney in use, and a second outer layer 211. This space 203 is filled with fluid coolant in use. A further cavity 231 is defined between the second outer layer 211 and the third external layer 230. This cavity 231 is inflatable and serves to facilitate the opening up of the bag within the abdominal cavity and also to provide a thermal insulation barrier between the kidney and surrounding organs in use. Tubular catheters 204a,b are provided from an external cooling system (not shown) along the shaft 202 portion of the device and into the space 203 between the first inner layer 210 and the second outer layer 211. These tubular catheters 204a,b deliver coolant to and remove coolant from this space 203. Semi-rigid plastic tubes (not shown in this Figure) are provided from an external source of air, through the shaft portion 202, and into the cavity 231 defined between the second outer layer 211 and the third external layer 230. A narrow channel 206 is defined near the border of the open end 252 of the sheath, along which a drawstring 220 is provided. A series of vertical welds 232 are provided between the second outer layer 211 and the third external layer 230, which compartmentalise the cavity 231. This serves to create a series of inflatable pockets which ensure uniform inflation of the cavity 232 and help maintain the overall shape of the device.

Finally, FIGS. 4a to 4f illustrate a series of locking means which are suitable for securing the drawstring of any of the embodiments of the laparoscopic cooling device of the invention. FIG. 4a depicts a toggle, which can be used to affix the drawstring in its pulled or tautened position. The locking means comprises a locking part 601 incorporating a first channel 602 through which both ends of the drawstring are slidingly located. The locking means also includes a spring loaded movable member 603 which is located within the locking part and is accessible from outside of the locking part via a button 604. The spring loaded movable member 603 incorporates a second channel 605 which overlaps with the first channel when the button is depressed, so that both ends of the drawstring are also slidingly located though the second channel. The spring loaded movable member 603 is biased towards a position whereby the first channel 602 does not overlap with the second channel 605, so that when at rest, the spring loaded movable member 603 clamps both ends of the drawstring between the spring loaded movable member 603 and the locking part to keep the drawstring pulled. In the event that the user wishes to release the drawstring, they simply depress the spring loaded movable member 603 whereupon the first 602 and second channels 605 overlap so that the drawstring can move freely through the locking mechanism.

FIG. 4b depicts a deformable rubber locking member, which is the preferred version for use with the laparoscopic cooling device. The deformable locking means comprises a simple rubber member 701 incorporating a transverse slit 702 therethrough which both ends of the drawstring are retained. In order to pull the drawstring to close the sheath, the user simply squeezes the rubber member 701 at either side of the slit 702 to remove the compressive force acting on the drawstring and allowing the drawstring to freely slide through the slit. To lock the drawstring in its pulled state, the user releases the rubber member. To release the drawstring to open the sheath once again, the user simply squeezes the rubber member 701 at either side of the slit 702, thereby allowing the drawstring to freely slide through the slit to open the sheath. To assist in the gripping action of the rubber member, the drawstring can be crimped or crenellations can be provided within the slit 702 of the rubber member 701. Alternatively, the simple rubber member incorporating the slit could be adapted so that one end of the drawstring is permanently fixed to the rubber member with the other end of the drawstring freely slidable through the slit.

FIG. 4c illustrates a Venetian blind-type of automatic locking mechanism, whereby both ends of the drawstring are clamped between a rotatably mounted toothed wheel 801 and a spring loaded wheel 802 which is biased towards the toothed wheel to clamp the drawstring, but which is movable away from the toothed wheel to bring it out of engagement with the drawstring to release the drawstring when required.

FIG. 4d illustrates a releasable cable tie type of locking arrangement, whereby a first end of the drawstring comprises a locking part 900 incorporating a channel 901 through which the second end 902 of the drawstring 120 may be slidably located. The drawstring is provided with a plurality of ridges (not shown) perpendicular to its length, along its length, and the locking part 900 includes a movable member 903 which is biased towards the channel 901 to abut against the ridges of the second end 902 of the drawstring 120 to keep the drawstring 120 pulled taut. In the event that the user wishes to release the drawstring 120 to open the sheath 101 once again, they press the movable member 903 to bring it out of engagement with the second end 902 of the drawstring 120 to thereby allow the second end 902 of the drawstring 120 to freely slide through the channel 901 and as a consequence open the sheath 101. With this embodiment, a thin, strong and flexible material may not be an ideal material for the drawstring 120 since it would not easily lend itself to the formation of ridges. In view of this, the inventors envisage a thicker and more rigid material for the drawstring if this embodiment of the locking mechanism were to be used. Alternatively, a thin, strong and flexible material still could be used for the drawstring 120, but with a thicker and more rigid ridged material being attached to a second end 902 of the drawstring 120, and the locking part 900 being attached to a first end of the thin and flexible drawstring 120.

FIG. 4e illustrates a ratchet and pawl type of locking mechanism. In this embodiment, both the ends of the drawstring (not shown) are wedged between a rotatably mounted ratchet wheel 400 having a plurality of teeth 401 around its periphery and a spring loaded pawl 402 which is biased towards the ratchet wheel 400 so that it selectively engages the gaps between the teeth 401 of the ratchet wheel 400, but which is movable away from the ratchet wheel 400 by means of a release member (not shown), when required. The ratchet and pawl arrangement and in particular the configuration of the teeth 401 ensures that the ratchet wheel 400 can rotate in one direction only and the drawstring can therefore be pulled in one direction only, for example in a direction such as to draw the drawstring. In order to release the drawstring and open the sheath once again, the user operates the release member to move the pawl 402 out of engagement with the ratchet wheel 400.

FIG. 4f illustrates a simple cuff type of locking mechanism. The cuff comprises a tubular member 500 which is disposed towards the near end of the sheath. Both ends of the drawstring (not shown) are able to locate inside of the tubular member 500. The interior of the tubular member 500 comprises a plurality of ridges 501 which maintain a frictional hold on the ends of the drawstring. The locking mechanism further includes a grasping tag (not shown) disposed at the far end of the sheath 101. The tubular member 500 is substantially tightly disposed around the drawstring to securely hold the two ends of the drawstring in place. In order to pull the drawstring, the user would simply hold onto the tubular member 500 and pull the drawstring as required to close the opening in the sheath. In order to open the sheath once again, the user would retain their hold on the tubular member 500 but additionally hold onto the tab at the far end of the sheath using a second grasping device, and pull on the tab, to once again overcome the frictional hold of the ridges 501 and thereby open the sheath.

Preferably, any locking mechanism used should be automatic, which means that the locking mechanism should activate of its own accord when the user stops pulling the drawstring to maintain the drawstring in its drawn position.

All of the above described locking mechanisms provide the advantage that they may be disposed outside of the patient's body and are therefore easily operable by a user. Alternatively, any of the above described locking mechanisms described above could be operated using a grasping device typically used during laparoscopic surgery. The simple rubber member version of the locking mechanism has the particular advantage that it is simple to use, and is inexpensive to manufacture. Moreover, all of the above described locking mechanisms facilitate the locking of the drawstring at any convenient point.

Whilst in the described and depicted embodiments the shaft portion is fixed to the sheath, alternative embodiments in which the shaft portion is not fixed to the sheath, or in which a shaft portion is not provided, can be envisaged.

It will be evident that various modifications and improvements could be made to the above-described device and methods within the scope of the invention. For example, the above description is written in the context of a laparoscopic kidney cooling device for use during partial renal nephrechtomy for the purposes of illustration of performance of the invention. However, the methods and apparatus apply equally to alternative body organs, such as the liver or spleen, and to alternative surgical procedures. Furthermore, it will be understood that the device can be provided in alternative sizes and shapes to ensure compatibility with differing body organs. The invention also contemplates devices of different sizes, for example suitable for paediatric and adult use.

Further modifications may be made without departing from the scope of the invention herein intended.

1-74. (canceled)

75. A laparoscopic cooling device for lowering the temperature of an organ comprising:
a sheath, wherein walls of the sheath comprise a first inner layer and a second outer layer which define a space therebetween; and
at least one coolant transport tube adapted to transport coolant into the space defined by the first inner layer and the second outer layer;
wherein at least one of the layers is deformable to an extent that it will conform to a surface of an organ when the space defined by the first inner layer and the second outer layer contains coolant.

76. A laparoscopic cooling device as claimed in claim 75 further comprising a shaft portion.

77. A laparoscopic cooling device as claimed in claim 75 wherein the walls of the sheath comprise a third layer.

78. A laparoscopic cooling device as claimed in claim 77 wherein the third layer is located external to the second outer layer and defines one or more cavities therebetween.

79. A laparoscopic cooling device as claimed in claim 78 wherein the one or more cavities between the third layer and the second outer layer are inflatable when air is provided thereto.

80. A laparoscopic cooling device as claimed in claim 77 wherein the third layer is connected to the second outer layer via a series of vertical welds.

81. A laparoscopic cooling device as claimed in claim 80 wherein the vertical welds are discontinuous.

82. A laparoscopic cooling device as claimed in claim 75 wherein the sheath is substantially rectangular in shape.

83. A laparoscopic cooling device as claimed in claim 75 wherein the sheath comprises an elongate tail portion.

84. A laparoscopic cooling device as claimed in claim 75 wherein the sheath comprises a bag having a first open end, whereby in use, the bag surrounds the organ.

85. A laparoscopic cooling device as claimed in claim 84 wherein the first open end lies in a plane perpendicular to a longitudinal axis of the laparoscopic cooling device.

86. A laparoscopic cooling device as claimed in claim 75 further comprising a plurality of coolant transport tubes.

87. A laparoscopic cooling device as claimed in claim 86 further comprising a first coolant transport tube and a second coolant transport tube, wherein the first coolant transport tube is configured to deliver the coolant into the space between the first inner layer and the second outer layer and the second coolant transport tube is configured to remove the coolant from the space.

88. A laparoscopic cooling device as claimed in claim 86 wherein the coolant transport tubes are integrated into the second outer layer of the sheath.

89. A laparoscopic cooling device as claimed in claim 86 wherein in use a supply of the coolant is delivered into the space and the coolant currently in the space is removed such that a constant cooling effect is provided by the sheath.

90. A laparoscopic cooling device as claimed in claim 86 wherein the coolant transport tubes are formed of a flexible material.

91. A laparoscopic cooling device as claimed in claim 86 wherein the coolant transport tubes are tubular catheters.

92. A laparoscopic cooling device as claimed in claim 90 wherein the flexible coolant transport tubes formed of the flexible material are integrated into the second outer layer of the sheath.

93. A laparoscopic cooling device as claimed in claim 86 wherein the coolant transport tubes are connected to an external cooling system.

94. A laparoscopic cooling device as claimed in claim 93 wherein the external cooling system is adapted so that a cooling fluid flows in a closed loop.

95. A laparoscopic cooling device as claimed in claim 93 wherein the external cooling system comprises at least one of a heat exchanger and a pump, a flow rate adjustment module, and a temperature monitoring element.

96. A laparoscopic cooling device as claimed in claim 75 wherein a narrow channel is provided at a border of an open end of the sheath.

97. A laparoscopic cooling device as claimed in claim 96 wherein the narrow channel is defined by a seam connecting the first inner layer and the second outer layer of the sheath to define a pathway which encircles the open end of the sheath.

98. A laparoscopic cooling device as claimed in claim 75 further comprising at least one manoeuvring device for manoeuvring the sheath to surround the organ of interest.

99. A laparoscopic cooling device as claimed in claim 75 wherein the sheath comprises at least one rigid or semi-rigid spine, the rigid or semi-rigid spine comprising a plastic rod.

100. A laparoscopic cooling device as claimed in claim 78 wherein the sheath comprises at least one rigid or semi-rigid tube, wherein the at least one rigid or semi-rigid tube is provided within one of the space defined by the first inner layer and the second outer layer and the cavity defined by the third layer and the second outer layer.

101. A laparoscopic cooling device as claimed in claim 100 wherein the at least one rigid or semi-rigid tube is configured to form a rigid spine of the sheath.

102. A laparoscopic cooling device as claimed in claim 100 wherein the at least one rigid or semi-rigid tube is one of a narrow plastic tube or tubes and a narrow brass tube.

103. A laparoscopic cooling device as claimed in claim 100 wherein the at least one rigid or semi-rigid tube at least one of extends from a shaft portion of the laparoscopic cooling device to a narrow channel at an edge of the sheath, and enters a narrow passage at a border of the sheath via an access point in a seam joining the first inner layer and the second outer layer.

104. A laparoscopic cooling device us claimed in claim 103 wherein a coupling or couplings form a seal between the at least one rigid or semi-rigid tube and its point of entry to the narrow passage at the border of the sheath to create an airtight passageway.

105. A laparoscopic cooling device as claimed in claim 75 further comprising at least one closing device for closing an open end of the sheath to surround the organ, wherein the at least one closing device is operable or partially operable outside of a patient's body.

106. A laparoscopic cooling device as claimed in claim 105 wherein the at least one closing device comprises a drawstring, the drawstring further comprising at least one deformable locking device, and the drawstring provided via at least one of within a narrow channel at a border of the sheath, encircling the narrow channel at the border of the sheath and along a tail portion of the sheath so that the end(s) of the drawstring are provided outside of a patient's body when in use, and within at least one semi-rigid tube or tubes.

107. A laparoscopic cooling device as claimed in claim 75 further comprising at least one opening device to facilitate an opening out of an open end of the sheath inside a patient's body, wherein the at least one opening device comprises a provision of at least one of CO₂ and air to an airtight passageway.

108. A laparoscopic cooling device as claimed in claim 75 further comprising at least one insertion device for facilitating an insertion of the laparoscopic cooling device through an introducer or trocar, wherein the at least one insertion device is a rigid overtube made in situ by rolling a sheet of stiff material around the sheath.

109. A laparoscopic cooling device as claimed in claim 75 further comprising at least one reversible partition device to partition the sheath when in position surrounding the organ, wherein the at least one partition device comprises one of a rip cord and a zip.

110. A laparoscopic cooling device as claimed in claim 75 wherein the organ is a kidney.

111. A laparoscopic cooling device as claimed in claim 75 wherein the laparoscopic cooling device is for use during and/or before a laparoscopic partial nephrectomy.

112. A laparoscopic cooling device as claimed in claim 75 further comprising a thermocouple or thermocouples, wherein at least one of a junction of the thermocouple or thermocouples provided at a surface of the first inner layer of the sheath which comes into contact with the organ, the junction is integrated into the sheath, the thermocouple or thermocouples are provided externally as part of a heat exchanger system, and the junction is provided on a coolant transport tube.

113. A laparoscopic cooling device as claimed in claim 75 further comprising markings to aid with depth perception.

114. A method of cooling an organ for surgery comprising:
inserting a sheath into a body cavity, wherein walls of the sheath comprise a first inner layer and a second outer layer which define a space therebetween;
employing at least one opening device to facilitate an opening out of an open end of the sheath inside the body cavity;
manipulating the sheath to surround an organ of interest;
securing the sheath around the organ of interest; and
providing coolant to the sheath;
wherein at least one coolant transport tube is adapted to transport coolant into the space defined by the first inner layer and the second outer layer;
wherein at least one of the layers is deformable to the extent that it will conform to a surface of the organ of interest when the space defined by the first inner layer and the second outer layer contains coolant.

115. A method of cooling an organ for surgery as claimed in claim 114 further comprising:
arranging the sheath into a tight bundle before insertion into the body cavity.

116. A method of cooling an organ for surgery as claimed in claims 115 further comprising:
wrapping a stiff sheet around the tight bundle to facilitate insertion into a trocar or introducer.

117. A method of cooling an organ for surgery as claimed in claim 114 comprising:
providing air to the sheath to cause it to inflate.

118. A method of cooling an organ for surgery as claimed in claim 114 comprising:
providing CO₂ to the sheath to cause it to inflate.

119. A method of cooling an organ for surgery as claimed in claim 114 further comprising:
securing the sheath around the organ of interest using a drawstring.

120. A method of cooling an organ for surgery as claimed in claims 114 further comprising:
monitoring a temperature of the organ of interest during a surgery.

121. A method of cooling an organ for surgery as claimed in claim 114 further comprising:
monitoring a temperature of the coolant during a surgery.
